(19) United States
(12) Patent Application Publication
(10) Pub. No.: US 2020/0303070 A1
(43) Pub. Date: Sep. 24, 2020

(54) CONTROL PROCESSES FOR MICROORGANISM-RELATED CHARACTERIZATION PROCESSES

(71) Applicant: PSOMAGEN, INC., Rockville, MD (US)

(72) Inventors: Zachary Apte, San Francisco, CA (US); Jessica Richman, San Francisco, CA (US); Daniel Almonacid, San Francisco, CA (US); Patricia Vera, San Francisco, CA (US); Elisabeth M. Bik, San Francisco, CA (US); Amanda Morton, San Francisco, CA (US); Tomas Norambuena, San Francisco, CA (US); Rodrigo Ortiz, San Francisco, CA (US)

(21) Appl. No.: 16/649,228

(22) PCT Filed: Nov. 6, 2018
(86) PCT No.: PCT/US2018/059488

§ 371 Date: Mar. 20, 2020

Related U.S. Application Data
(60) Provisional application No. 62/582,172, filed on Nov. 6, 2017.
(60) Provisional application No. 62/671,435, filed on May 15, 2018.

(51) Int. Cl. G16H 50/20; G01N 1/28; C12Q 1/689; G16B 10/00; G16H 10/40; G16H 70/60; G16H 50/70; G16H 20/00

(52) U.S. Cl. CPC G16H 50/20; G01N 1/286; C12Q 1/689; G16H 20/00; G16H 10/40; G16H 70/60; G16H 50/70; G16B 10/00

(57) ABSTRACT

Embodiments of a method and/or system, such as for improving a microorganism-related characterization process, can include: preparing a set of control samples (e.g., from an individual specimen); determining one or more reference microorganism-related parameters (e.g., cutoff reference ranges of relative abundance for a set of microorganism taxa) based on one or more control samples of the set of control samples; and determining one or more variability parameters for the microorganism-related characterization based on the one or more reference microorganism-related parameters and one or more control sample characterizations for one or more control samples of the set of control samples.

### CROSS-REFERENCE TO RELATED APPLICATIONS

This application claims the benefit of U.S. Provisional Application Ser. No. 62/582,172 filed 6 Nov. 2017, and U.S. Provisional Application Ser. No. 62/671,435 filed 15 May 2018, each of which are incorporated in their entirety herein by this reference.

### TECHNICAL FIELD

The disclosure generally relates to genomics and microbiology.

### BACKGROUND

Sequencing of the 16S rRNA (“16S”) gene can be used for microbiome analysis of biological specimens and/or suitable samples in order to determine which microorganisms are present in a biological sample, such as stool or soil. The 16S gene includes highly variable DNA sequences that can be used to identify microorganisms (e.g., taxa corresponding to the microorganisms, etc.). For example, bacterial and archaeal genomes include one or more copies of this gene, and the DNA sequences of this gene differ between microbial groups (e.g., microorganism taxa, etc.). The DNA sequence of this gene therefore can be used to determine to which taxon (e.g., genus, species, etc.) a bacterium, archaeon, and/or suitable microorganism belongs. In an example, databases include 16S sequences corresponding to different microorganism taxa (e.g., various microbial species and strains).

Microbiome analysis based on the 16S gene and/or other suitable data can include absolute or relative abundances of different microorganism taxa (e.g., bacteria, archaea, viruses, eukaryotic microbes, etc.). Observed microbiome composition diversity (e.g., microbiome analysis profiles, etc) can be dependent on a wide range of factors, such as specimen storage conditions, DNA extraction methods, marker gene amplification primers and techniques, sequencing methods, and/or bioinformatics pipeline tools. Reproducibility can be affected (e.g., where increased variability is present) by random bias such as from enzymatic amplification of the 16S gene by polymerase chain reaction (“PCR”). Additionally, reproducibility can be affected by use of PCR machines, reagent batches, operators, and/or other suitable aspects. Negative effects on reproducibility can bias microorganism-related characterizations, such as determinations of absolute and/or relative abundances of microorganism taxa and/or suitable determinations of microbiome composition, microbiome function, and/or any suitable microorganism-related characterization.

### BRIEF DESCRIPTION OF THE FIGURES

FIG. 1 includes a flowchart representation of variations of an embodiment of a method;

FIG. 2 includes a flowchart representation of variations of an embodiment of a method;

FIG. 3 includes a specific example of a graph representation of relative abundances for a set of control samples prepared from an individual specimen, and for a set of samples from different specimens;

FIG. 4 includes a specific example of a graph representation of relative abundances for a set of control samples prepared from a stool specimen;

FIG. 5 includes a specific example of a graph representation of reproducibility of microbial analysis for a set of control samples prepared from an individual specimen, and for a set of samples from different specimens.

### DESCRIPTION OF THE EMBODIMENTS

The following description of the embodiments is not intended to limit the embodiments, but rather to enable any person skilled in the art to make and use.

### 1. Overview

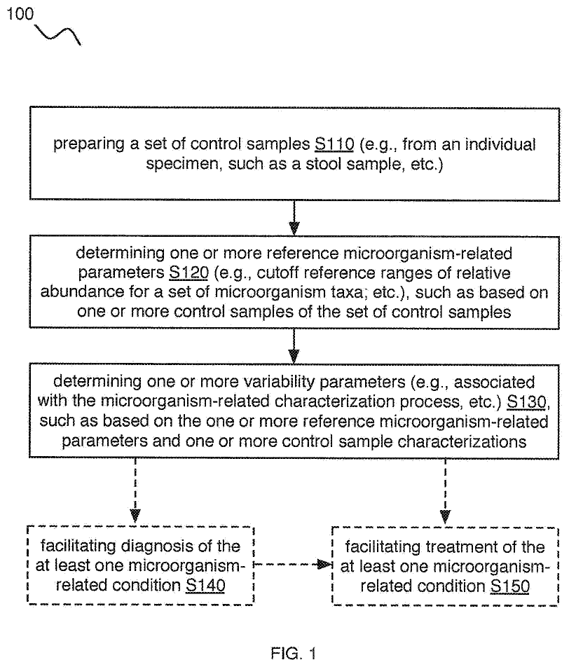

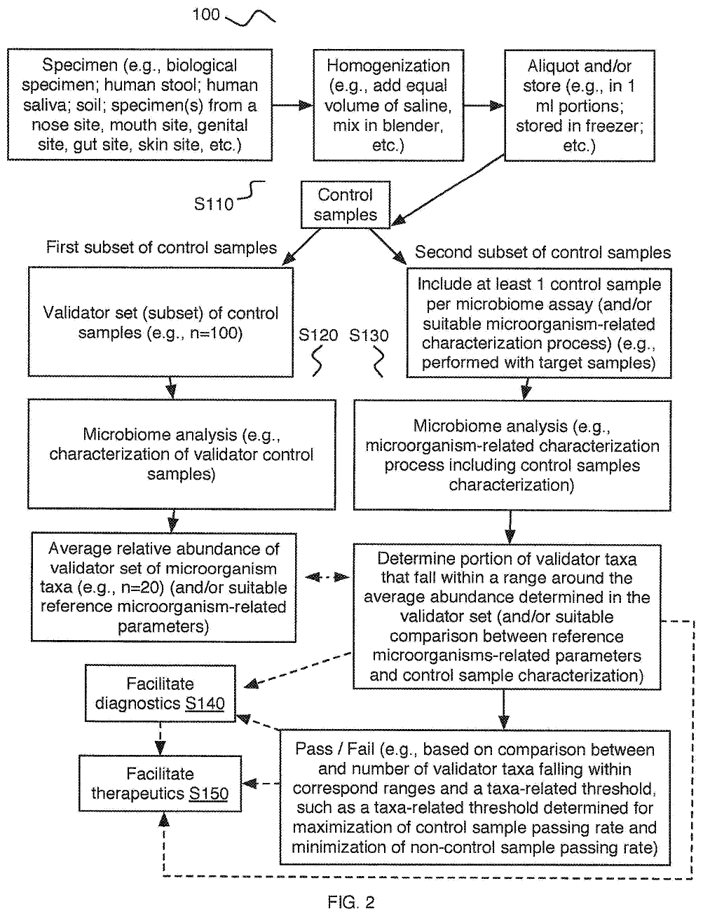

As shown in FIGS. 1-2, embodiments of a method 100 (e.g., for improving one or more microorganism-related characterization processes, such as in relation to quality, reproducibility, bias reduction, etc.) can include: preparing a set of control samples S110 (e.g., from an individual specimen, such as a stool sample, etc.); determining one or more reference microorganism-related parameters S120 (e.g., cutoff reference ranges of relative abundance for a set of microorganism taxa; etc.) based on one or more control samples of the set of control samples; and/or determining one or more variability parameters (e.g., associated with the microorganism-related characterization process, etc.) S130 based on the one or more reference microorganism-related parameters and one or more control sample characterizations for one or more control samples of the set of control samples.

In a specific example, the method 100 (e.g., for improving a microorganism-related characterization process, etc.) can include: preparing a set of control samples; determining a control sample characterization for at least one control sample of the set of control samples, based on the microorganism-related characterization process with the at least one control sample and a target sample; and/or determining a variability parameter for the microorganism-related characterization process based on a comparison between the control sample characterization and a reference microorganism-related parameter determined from processing operations associated with the microorganism-related characterization process.

In a specific example, the method 100 (e.g., for improving a microorganism-related characterization process, etc.) can include preparing a set of control samples from an individual specimen (e.g., an individual stool sample; etc.); determining a set of reference microorganism abundance parameters based on first processing operations with a first subset of control samples from the set of control samples, where the first processing operations are associated with the microorganism-related characterization process; determining a control sample characterization for a second subset of control samples from the set of control samples, based on the microorganism-related characterization process with the second subset of control samples and a target sample from a user; and/or determining a variability parameter for the microorganism-related characterization process (e.g., classifying a microbiome assay run as passing or failing; etc.) based on a comparison between the control sample characterization and the set of reference microorganism abundance parameters.

In a specific example, a homogenized biological control specimen is prepared and aliquoted to generate a set of control samples; a first subset of the set of control samples can be used as validator control samples to determine average and variation of relative abundance of a set of validator microorganism taxa; and a second subset of the set of control samples (e.g., from the same batch; etc.) can be used to validate the quality of suitable microorganism-related characterization processes (e.g., involving target samples to be characterized, such as in relation to one or more microorganism-related conditions; etc.).

Embodiments of the method 100 and/or system can function to improve reproducibility of microorganism-related characterizations, such as by accurately monitoring and capturing microbiome composition (e.g., microbiome profile) variability (e.g., small variation; large variation; etc.) of one or more control samples (e.g., included in every assay; using the magnitude of the variations as an indicator as to the quality of the assay and/or experimental process; etc.). In specific examples, the quality of microorganism-related characterizations (e.g., microbiome profiles; relative abundance of microorganism taxa, such as in relation to microorganism composition diversity; etc.) of control samples (e.g., control specimens) can be determined and/or applied in determining the quality of a microorganism-related experimental process (e.g., assay; etc.). In a specific example, reproducibility (e.g., degree of variability; etc.) can be monitored at all and/or any number of stages of the microbiome characterization processes, such as in order to measure and quantify variability associated with microbiome characterization. In a specific example, an assay can be validated (e.g., pass, etc.) based on a variability parameter indicating a deviation below a threshold (e.g., a sufficiently small deviation, etc.), or the assay can be failed based on the variability parameter indicating a deviation above a threshold (e.g., a sufficiently large deviation, etc.). In specific examples, challenges with microbiome characterization interpretation (e.g., challenges with interpreting microbiome characterization metrics as positive or negative values; using relative abundances of a variety of microorganism taxa; etc.) can be overcome, such as for improving reproducibility of microorganism-related characterizations. However, embodiments of the method 100 and/or system can include any suitable functionality. In specific examples, embodiments can function to monitor the quality and/or reproducibility of the different steps of the assay, such as but not limited to: DNA extraction, amplification, sequencing, bioinformatic analysis, and any other suitable analysis for present technology can be applied.

Additionally or alternatively, embodiments of the method 100 can include facilitating diagnostics S140 (e.g., based on the one or more variability parameters; etc.); facilitating therapeutics S150 (e.g., based on the one or more variability parameters; etc.); and/or any other suitable processes. For example, variability parameters characterizing one or more microorganism-related characterization processes (e.g., microbiome assays) can be used in determining whether to use (and/or to what degree to use; and/or in which manner to use; etc.) such microorganism-related characterization processes (e.g., discarding a microbiome assay run in response to the variability parameter indicating a failing metric for the microbiome assay run; etc.). In variations, the method 100 can include determining a therapy for the user for a microorganism-related condition based on the variability parameter for the microorganism-related characterization process; and facilitating provision of the therapy to the user, such a where facilitating provision of the therapy can include providing the therapy to the user. In variations, one or more microorganism-related characterization processes can include one or more microbiome assays associated with diagnostics for one or more microorganism-related conditions, such as where determining the variability parameter can be for the one or more microbiome assays. However, facilitating diagnostics S140 and/or facilitating therapeutics S150 can be performed in any suitable manner.

Embodiments of the method 100 and/or system can additionally or alternatively generate and/or promote (e.g., provide; present; notify regarding; etc.) characterizations (e.g., diagnoses, etc.) and/or therapies for one or more microorganism-related conditions (e.g., based on variability parameters, microorganism-related parameters, reference microorganism-related parameters, control sample characterizations, etc.), such as facilitating diagnostics S140 and/or facilitating therapeutics S150 for one or more microorganism-related conditions.

Microorganism-related conditions can include one or more of: diseases, symptoms, causes (e.g., triggers, etc.), disorders, associated risk (e.g., propensity scores, etc.), associated severity, behaviors (e.g., caffeine consumption, alcohol consumption, sugar consumption, habits, diets, etc.), and/or any other suitable aspects associated with microorganism-related conditions. Microorganism-related conditions can include one or more disease-related conditions, which can include any one or more of: gastrointestinal-related conditions (e.g., irritable bowel syndrome, inflammatory bowel disease, ulcerative colitis, celiac disease, Crohn's disease, bloating, hemorrhoidal disease, constipation, reflux, bloody stool, diarrhea, etc.); allergy-related conditions (e.g., allergies and/or intolerance associated with wheat, gluten, dairy, soy, peanut, shellfish, tree nut, egg, etc.); locomotor-related conditions (e.g., gout, rheumatoid arthritis, osteoarthritis, reactive arthritis, multiple sclerosis, Parkinson's disease, etc.); cancer-related conditions (e.g., lymphoma; leukemia; blastoma; germ cell tumor; carcinoma; sarcoma; breast cancer; prostate cancer; basal cell cancer; skin cancer; colon cancer; lung cancer; cancer conditions associated with any suitable physiological region; etc.); cardiovascular-related conditions (e.g., coronary heart disease, inflammatory heart disease, valvular heart disease, obesity, stroke, etc.); anemia conditions (e.g., thalassemia; sickle cell; pernicious; fanconi; hemolytic; aplastic; iron deficiency; etc.); neurological-related conditions (e.g., ADHD, ADD, anxiety, Asperger's syndrome, autism, chronic fatigue syndrome, depression, etc.); autoimmune-related conditions (e.g., Sprue, AIDS, Sjogren's, Lupus, etc.); endocrine-related conditions (e.g., obesity, Graves' disease, Hashimoto's thyroiditis, metabolic disease, Type I diabetes, Type II diabetes, etc.); skin-related conditions (e.g., acne, dermatomyositis, eczema, rosacea, dry skin, psoriasis, dandruff, photosensitivity, rough skin, itching, flaking, scaling, peeling, fine lines or cracks, gray skin in individuals with dark skin, redness, deep cracks such as cracks that can bleed and lead to infections, itching and scaling of the skin in the scalp, oily skin such as irritated oily skin, skin sensitivity to products such as hair care products, imbalance in scalp microbiome, etc.); Lyme disease conditions; communication-related conditions; sleep-related conditions; metabolic-related conditions; weight-related conditions; pain-related conditions; genetic-related conditions; chronic disease; and/or any other suitable type of disease-related conditions. In variations, microorganism-related conditions can include one or more women's health-related conditions (e.g., reproductive system-related conditions; etc.). In variations, microorganism-related conditions can include mosquito-related conditions, such as conditions including and/or associated with mosquito bites, malaria, and/or other suitable conditions associated with mosquitoes. In variations, microorganism-related conditions can include insect-related conditions associated with any suitable insect bites and/or insects.

In variations, control sample preparation and or usage, determination of reference microorganism-related parameters (e.g., refining of microorganism-related parameters; etc.), determination of variability parameters, and/or any suitable portions of embodiments of the method 100 can be performed over time (e.g., at time intervals, any suitable amount of frequency and time, etc.), such as to monitor, react to, facilitate, and/or otherwise process microorganism-related characterizations and/or control sample characterizations, such as in relation to monitoring, diagnostics, and/or therapeutics for one or more users for one or more microorganism-related conditions. In specific examples, control sample usage and/or determination of variability parameters can be performed for one or more specific time periods (e.g., a time period pre-, during, and/or post-time period associated with one or more microorganism-related conditions for a user; etc.) and/or regularly at specified time intervals. Additionally or alternatively, any suitable portions of embodiments of the method 100 (e.g., control sample usage; determination of variability parameters; etc.) can be performed at one or more points of care for one or more users (e.g., at an individual's home and/or at suitable locations; at non-laboratory locations; at non-care provider locations; etc.)

Additionally or alternatively, embodiments of the method 100 and/or system can function to improve identification of microbiome features supplemental features (e.g., derived from supplemental data, etc.), and/or other suitable data associated with (e.g., positively correlated with, negatively correlated with, etc.) one or more microorganism-related conditions, such as based on using variability parameters for improving microorganism-related characterization processes. In examples, microorganism-related conditions can be associated with at least one or more of microbiome composition (e.g., microbiome composition diversity, etc.), microbiome function (e.g., microbiome functional diversity, etc.), and/or other suitable microbiome-related aspects.

In variations, embodiments of the method 100 and/or system can determine variability parameters and/or suitable data described herein for one or more microorganism-related characterization processes (e.g., for determining one or more microorganism-related characterizations and/or therapies associated with one or more microorganism-related conditions, etc.) described in and/or analogous U.S. application Ser. No. 15/707,907 filed 18 Sep. 2017, which is herein incorporated in its entirety by this reference.

In variations, samples (e.g., described herein; control samples; target samples; etc.), microorganism-related conditions, microorganism-related characterization processes, microorganism-related parameters, variability parameters, and/or any suitable components (e.g., described herein; etc.) can be derived from, collected form, and/or otherwise associated with one or more body sites including at least one of a gut body site (e.g., corresponding to a body site type of a gut site; such as a stool sample; etc.), a skin body site (e.g., corresponding to a body site type of a skin site), a nose body site (e.g., corresponding to a body site type of a nose site), a mouth body site (e.g., corresponding to a body site type of a mouth site), a genitals body site (e.g., corresponding to a body site type of a genital site) and/or any suitable body sites located at any suitable part of the body.

In variations, portions of embodiments of the method 100 can be repeatedly performed in any suitable order and/or any suitable components of embodiments of the system can be repeatedly applied, such as to improve any suitable portions of embodiments of the method 100 and/or any suitable components of embodiments of the system. In variations, the method 100 can be repeatedly performed to enable refining of reference microorganism-related parameters, models (e.g., variability parameter models, etc.), microorganism-related characterization processes, control sample preparation processes, and/or any suitable aspects.

Data described herein (e.g., variability parameters, microorganism-related parameters, reference microorganism-related parameters, control sample characterizations, microorganism-related characterizations, data associated with control sample preparation, etc.) can be associated with any suitable temporal indicators (e.g., seconds, minutes, hours, days, weeks, months, years, etc.) including one or more: temporal indicators indicating when the data and/or components were collected (e.g., temporal indicators indicating when a sample was collected; sampling time; temporal indicators indicating when a specimen was collected; etc.), determined, transmitted, received, and/or otherwise processed; temporal indicators providing context to content described by the data (e.g., temporal indicators associated with control samples, with variability parameters, etc.); changes in temporal indicators (e.g., changes in microbiome over time; such as in response to receiving a therapy; changes in variability parameters over time; latency between sample collection, sample analysis, provision of a microorganism-related characterization or therapy to a user's; and/or suitable portions of embodiments of the method 100; etc.); and/or any other suitable indicators related to time.

Additionally or alternatively, parameters, metrics, inputs, outputs, and/or other suitable data (e.g., described herein, etc.) can be associated with value types including: scores (e.g., variability scores, quality scores, propensity scores, feature relevance scores, correlation scores; covariance scores; microbiome diversity scores, severity scores, etc.); individual values, aggregate values, (e.g., average reference microorganism-related parameters, etc.), binary values (e.g., classifications of a microbiome assay as passing or failing; etc.), relative values (e.g., relative taxonomic group abundance, relative microbiome function abundance, relative feature abundance, etc.), classifications (e.g., for characterizing a microorganism-related characterization process; microorganism-related condition classifications and/or diagnoses for users; etc.), confidence levels, identifiers, values along a spectrum, and/or any other suitable types of values. Any suitable types of data described herein can be used as inputs (e.g., for different analytical techniques, models, and/or other suitable components described herein), generated as outputs (e.g., of different analytical techniques, models, etc.), and/or manipulated in any suitable manner for any suitable components associated with the method 100 and/or system.

One or more instances and/or portions of embodiments of the method 100 and/or processes described herein can be performed asynchronously (e.g., sequentially), concurrently (e.g., parallel data processing; parallel sample processing; parallel control sample preparation; multiplex sample processing; performing sample processing and analysis for substantially concurrently evaluating a panel of microorganism-related conditions and/or users; computationally determining variability parameters for a plurality of microorganism-related characterization processes; such as concurrently on different threads for parallel computing to improve system processing ability; etc.), in temporal relation (e.g., substantially concurrently with, in response to, serially, prior to, subsequent to, etc.) to a trigger event (e.g., performance of a portion of embodiments of the method 100), and/or in any other suitable order at any suitable time and frequency by and/or using one or more instances of the system, components, and/or entities described herein.

Portions of embodiments of the method 100 (e.g., determining control sample characterizations, determining reference microorganism-related parameters; etc.) and/or system can use, apply, and/or otherwise be associated with one or more sequencing systems (e.g., next-generation sequencing systems, sequencing systems for targeted amplicon sequencing, sequencing-by-synthesis techniques, capillary sequencing technique, Sanger sequencing, pyrosequencing techniques, nanopore sequencing techniques, etc.) for sequencing one or more samples (e.g., sequencing microorganism nucleic acids from the biological samples; control samples; target samples; etc.). Sequencing systems (e.g., next-generation sequencing platforms, etc.) can include any suitable sequencing systems (e.g., sequencing platforms, etc.) for one or more of high-throughput sequencing (e.g., facilitated through high-throughput sequencing technologies; massively parallel signature sequencing, Polony sequencing, 454 pyrosequencing, Illumina sequencing, SOLiD sequencing, Ion Torrent semiconductor sequencing, DNA nanoball sequencing, Heliscope single molecule sequencing, Single molecule real time (SMRT) sequencing, Nanopore DNA sequencing, etc.), any generation number of sequencing technologies (e.g., second-generation sequencing technologies, third-generation sequencing technologies, fourth-generation sequencing technologies, etc.), amplicon-associated sequencing (e.g., targeted amplicon sequencing), sequencing-by-synthesis, tunnelling currents sequencing, sequencing by hybridization, mass spectrometry sequencing, microscopy-based techniques, and/or any suitable sequencing technologies.

However, the method 100 and/or system can be configured in any suitable manner.

### 2.1 Preparing Control Samples.

Embodiments of the method 100 can include preparing a set of control samples S110, which can function to generate control samples for use in determining reference microorganism-related parameters and/or control sample characterizations, such as in relation to determining variability parameters associated with (e.g., for, etc.) one or more microorganism-related characterizations. In a specific example, a biological specimen can be homogenized and aliquoted to generate a set of control samples (e.g., a set of replicate samples) for use in a plurality of microbiome assays (e.g., validation assays for determining reference microorganism-related parameters; experimental assays for determining microorganism-related characterizations, such as in relation to diagnostics and/or therapeutics; etc.).

The set of control samples is preferably generated from an individual specimen (e.g., a single specimen, etc.). For example, preparing a set of control samples can include preparing a set of control samples from an individual specimen, and where preparing the set of control samples from the individual specimen can include homogenizing the individual specimen. A specimen can include any one or more of: a stool sample, a saliva sample, a soil sample, a sample from a human, a sample from an animal, a nature sample, samples from any suitable body site (e.g., skin site, mouth site, genital site, gut site, nose site, etc.), biological samples, non-biological samples, and/or any suitable types of specimens. For example, preparing the set of control samples from the individual specimen can include homogenizing the individual specimen in relation to microorganism content from the individual specimen; and aliquoting the homogenized individual specimen; and where the individual specimen can include a stool sample; and where homogenizing the individual specimen includes mixing the stool sample with a saline solution.

Additionally or alternatively, any suitable number of control samples can be generated from any suitable number of specimens.

Specimens preferably include microorganisms (e.g., from a set of taxa; from a taxon; etc.), but can additionally or alternatively include any suitable components.

Preparing control samples can include one or more of: generating (e.g., creating, etc.) control samples; sample processing (e.g., sample processing resulting in control samples; etc.); providing control samples (e.g., to a third party, such as for a third party's use in determining variability parameters for performed assays; etc.); and/or suitable processes for preparing control samples.

Preparing control samples preferably includes one or more homogenization processes. Homogenization processes preferably include homogenizing one or more specimens (e.g., in relation to homogenization across the specimen in relation to microorganism distribution across the sample, etc.)/, but can include homogenization processes for any suitable components (e.g., processed specimens; samples; etc.). In a specific example, homogenization can include homogenizing a specimen of a volume large enough to be included in a plurality of microbiome analysis assays (and/or suitable control sample characterization processes and/or microbiome characterization processes; etc.). In a specific example, preparing control samples can include homogenizing a human stool specimen (e.g., a complete bowel movement) based on mixing with an equal amount (e.g., equal volume) (and/or any suitable amount) of saline solution (and/or other suitable solutions) and mixing for a time period (e.g., 1 minute; any suitable time period; etc.), such as in a blender and/or using any suitable mixing mechanisms. However, homogenization processes can be performed in any suitable manner.

Preparing control samples preferably includes one or more aliquoting processes. Aliquoting processes are preferably performed on homogenized specimens, but can additionally or alternatively be performed on any suitable specimens with any suitable degree of homogenization and/or heterogenization. In a specific example, a homogenized human stool specimen (e.g., homogenized using homogenization processes described herein, etc) can be aliquoted into a plurality (e.g., any suitable number) of portions (e.g., hundreds of 1 to 50 ml portions, and/or portions of any suitable volume; etc.), and/or stored at suitable conditions and/or temperatures (e.g., a −20° C. and/or −80° C. freezer and/or any other suitable temperature-regulated environment; etc.), such as where the portions can be used as control samples.

Prepared control samples can be used for any suitable number and/or type of experimental assays and/or suitable experimental processes performed at any suitable time (e.g., control samples can be stored and preserved for use at any suitable time following preparation; etc.) by any suitable entities (e.g., by any suitable manual and/or automated operators; etc.).

Additionally or alternatively, preparing control samples (and/or any suitable portions of embodiments of the method 100 and/or system, etc.) can include any suitable sample processing techniques, including any one or more of: sequencing operations, alignment operations (e.g., sequencing read alignment; etc.), lysing operations, cutting operations, tagging operations (e.g., with barcodes; etc.), ligation operations, fragmentation operations, amplification operations (e.g., helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), etc.), purification operations, cleaning operations, homogenization processes, heterogenization processes, aliquoting processes, replicate sample preparation processes, suitable operations for sequencing library preparation, suitable operations for facilitating sequencing and/or downstream analysis, suitable sample processing operations, and/or any suitable sample- and/or sequence-related operations.

However, preparing control samples S110 can be performed in any suitable manner.

### 2.2 Determining a Reference Microorganism-Related Parameter.

Embodiments of the method 100 can include determining one or more reference microorganism-related parameters S120, which can function to determine one or more reference parameters for use in determining one or more variability parameters (e.g., based on comparing the reference parameters to values from a control sample characterization included in a microorganism-related characterization process (e.g., microorganism-related assay).

Reference microorganism-related parameters preferably include one or more microorganism abundance parameters. Microorganism abundance parameters can include any one or more of: relative abundance values (e.g., for a microorganism taxa in the context of a plurality of microorganism taxa present in one or more samples; for a microorganism taxa in the context of the microbiome present in one or more samples; etc.), absolute abundance values (e.g., for a microorganism taxa; absolute counts of microorganisms present in one or more samples; etc.); and/or any suitable types of abundance parameters. Additionally or alternatively, reference microorganism-related parameters can include any suitable microbiome composition parameters (e.g., microbiome composition data; microbiome composition features; microbiome composition diversity; etc.); microorganism function parameters (e.g., associated with microorganism function; microbiome functional data; microbiome functional features; microbiome functional diversity; etc.); and/or any suitable types of microorganism-related parameters. Reference microorganism-related parameters can be of any suitable forms, including any one or more of: ranges (e.g., a reference cutoff range for relative abundance of a given microorganism taxon; etc.), averages (e.g., average relative abundance for a given microorganism taxon; from which a range can be determined; etc.), medians, standard deviations (e.g., in relation to averages, for use in determining ranges; etc.), overall values (e.g., determined from individual values; etc.); absolute values (e.g., absolute counts for a microorganism taxon, etc.); changes in values (e.g., changes in relative abundance over time from a series of control samples collected over time; changes in any suitable parameters over time; changes in suitable parameters across experimental conditions, such as in relation to different operators, different experimental runs, and/or other suitable conditions; etc.); and/or any suitable forms of reference microorganism-related parameters.

Reference microorganism-related parameters can include a set of reference ranges (e.g., based on averages for relative abundance and variation around the averages, etc.) for a set of microorganism taxa (e.g., a reference range for each validator taxon to be used in comparisons with control sample characterizations associated with microorganism-related characterization processes for target samples; etc.). In a specific example, determining reference microorganism-related parameters can include: determining individual abundance parameters from a set of validation assays (e.g., each including at least one control sample, such as control samples generated from the same specimen; etc.), such as where, for each taxon of a set of validation taxa, a set of individual abundance parameters is determined (e.g., for each taxon, an individual abundance parameter is determined for each of the set of validation assays; etc.); determining, for each validator taxon, a mean abundance parameter (e.g., from averaging individual abundance parameters for the validator taxon; etc); and determining the reference microorganism-related parameters (e.g., ranges of abundance, such as ranges of relative abundance, for each of the validator taxa; etc.) based on the mean abundance parameters (e.g., ranges determined based on variation around the mean, such as in relation to standard deviation around the mean; etc.).

In a specific example, a plurality of control samples (e.g., 50 or 100 replicate specimen aliquots) can be used in one or more independent microbiome validation assays for determining individual abundance parameters for a set of taxa, such as where the individual abundance parameters can be used in determining mean abundance parameters, reference ranges, and/or suitable reference microorganism-related parameters.

In a specific example, a reference microorganism-related parameter can include a reference microorganism relative abundance parameter for a microorganism taxon, where the control, sample characterization (e.g., performed for a control sample ran with one or more target samples of a microorganism-related characterization process; etc.) can include a microorganism relative abundance parameter for the microorganism taxon, and where determining the variability parameter can include determining the variability parameters based on the comparison between the microorganism relative abundance parameter and the reference microorganism relative abundance parameter.

Reference range cutoffs, such as score cutoffs, (and/or any suitable determinations of thresholds and/or suitable reference microorganism-related parameters) can be determined based on maximizing the number of control samples from otherwise validated microbiome assays that pass, while minimizing the number of non-control samples to pass. Additionally or alternatively, reference range cutoffs and/or any suitable thresholds and/or suitable reference microorganism-related parameters can be determined based on any suitable criteria.

In a specific example, determining the set of reference microorganism abundance parameters can include determining a set of individual reference microorganism abundance parameters (e.g., for a set of taxa; by processing a set of control samples, where each control sample can result in a set of individual reference microorganism abundance parameters for the set of taxa; etc.) based on the first processing operations with the first subset of control samples (e.g., derived from an individual specimen; etc.); and determining a set of average reference microorganism abundance parameters based on the set of individual reference microorganism abundance parameters (e.g., determine an average for each taxon of the set of taxa; etc.), where determining the variability parameter can include determining the variability parameter based on the control sample characterization and the set of average reference microorganism abundance parameters (e.g., comparing microorganism abundance parameters of the control sample characterization to the set of average reference microorganism abundance parameters; etc.). In a specific example, determining the set of reference microorganism abundance parameters can include determining a set of reference microorganism abundance ranges (e.g., cutoff ranges for abundances for different taxa, etc.) based on the set of average reference microorganism abundance parameters, and where determining the variability parameter can include determining the variability parameter based on the comparison between the control sample characterization and the set of reference microorganism abundance ranges. In a specific example, determining the set of reference microorganism abundance ranges can include determining the set of reference microorganism abundance ranges for a set of validator microorganism taxa (e.g., a subset of taxa from a pool of potential taxa for which microbiome composition was determined; etc.), where the control sample characterization can include a set of microorganism abundance parameters for the set of validator microorganism taxa, and where determining the variability parameter can include, for each taxon of the set of validator taxa, determining whether a corresponding microorganism abundance parameter (e.g., for the taxon) of the set of microorganism abundance parameters is in (e.g., falls within, etc.) a corresponding reference microorganism abundance range (e.g., for the taxon) of the set of reference microorganism abundance ranges. In a specific example, determining a variability parameter can include determining a taxa-related score based on a number of taxa (e.g., from the set of validator taxa, etc.) with the corresponding microorganism abundance parameters in the corresponding reference microorganism abundance ranges; and determining the variability parameter based on a comparison between the taxa-related score and a taxa-related score threshold (e.g., determining a passing metric if the taxa-related score is at and/or exceeds the taxa-related score threshold; determining a failing metric if the taxa-related score is below the taxa-related score threshold; etc.). In a specific example, the method 100 can include determining the taxa-related score threshold based on a set of criteria associated with maximization of control sample passing rate and minimization of non-control sample passing rate.

In a specific example, the set of reference microorganism abundance ranges can include a set of reference microorganism relative abundance ranges, where the control sample characterization can include a set of microorganism relative abundance parameters, and where determining the variability parameter can include determining the variability parameter based on the comparison between the set of microorganism relative abundance parameters and the set of reference microorganism relative abundance ranges.

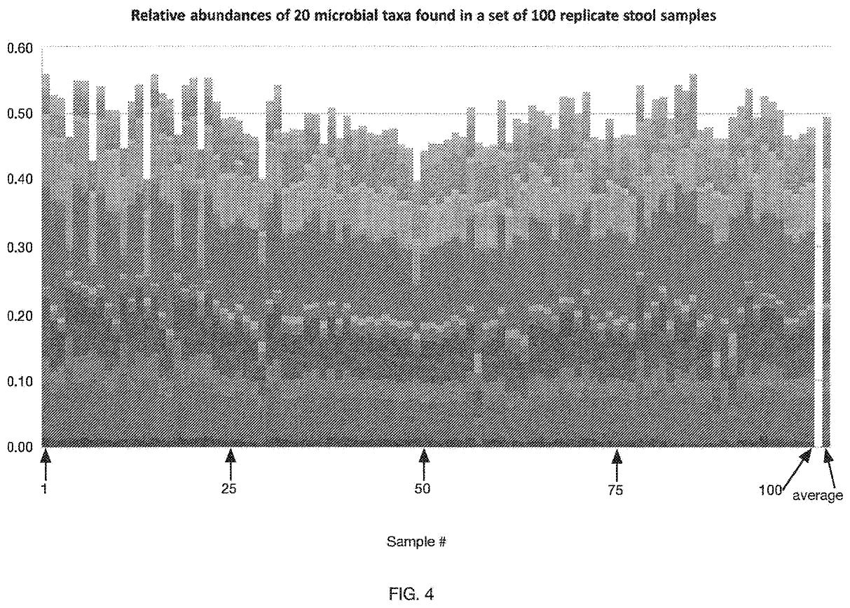

In a specific example, as shown in FIG. 4 (e.g., illustrating cumulative relative abundances and variation for 20 selected validator microbial taxa found in a set of 100 replicate stool control samples, where the set of 100 control samples analyzed in 100 independent microbiome assays, etc.), microorganism abundance parameters for a set of taxa can be analyzed (e.g., for abundance parameters; etc.) in relation to a predefined number of control samples (e.g., 100 replicate aliquots; a validator set of control samples; control samples generated from a single homogenized and aliquoted stool sample; dilutions of controls samples; etc.); where validator taxa can be determined based on the microorganism abundance parameters, such as selecting a predefined number of taxa with greatest abundance parameter values (e.g., selecting 20 most abundant taxa, such as bacterial taxa, for the validator taxa; etc.); and where the microorganism abundance parameters for the validator taxa can be used in determining the reference microorganism-related parameters (e.g., reference cutoff ranges; for use as reference in subsequent microbiome assays and/or suitable microorganism-related characterization processes, such as involving characterization of target samples; etc.), such as, for a microorganism-related characterization process (e.g., subsequent microbiome assay; etc.), a deviation from average (e.g., as shown in the right-most column in FIG. 4; etc.) for a control sample characterization (e.g., determined for one or more control samples used in the microorganism-related characterization process; etc.) can indicate quality (and/or reproducibility, and/or other suitable aspects) of the microorganism-related characterization process (e.g., where a large deviation can indicate a poor-quality run; etc.).

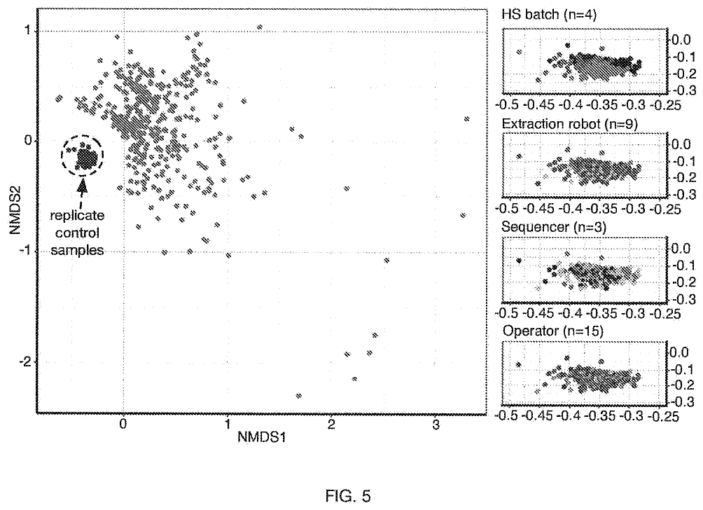

In examples (e.g., as shown in FIG. 5, etc.) validation for using a homogenized individual specimen for a set of control samples to be used in evaluating one or more microbiome-related characterization processes (e.g., microbiome assays) can be performed. In a specific example, as shown in FIG. 5, reproducibility can be tested for DNA extraction, amplification methods, and/or suitable sample processing operations, such as in a high-throughput laboratory setting. As shown in FIG. 5, 363 control samples (e.g., 363 aliquots), derived from a same, single, homogenized human stool specimen, can be prepared in four different batches, and each extracted in a different DNA extraction run; where each aliquot can be processed independently on a separate DNA extraction and PCR amplification run, using the same standard operating procedure executed by a rotating group of different operators; and where the relative abundances of the clinical genera in each of these 363 control samples can be compared with each other; where results can show overall microbiome profiles (e.g., relative abundance values; microbiome composition; etc.) of the control samples to be similar to each other; where beta-diversity analysis can show control samples clustered tightly together, irrespective of the operator, extraction robot, or sequencer; where a set of 400 stool samples from a subset of 897 different healthy subjects (e.g., as opposed to from a single specimen from a single subject; etc.) can show substantially different microbiome profiles (e.g., relative abundance values; microbiome composition; etc.), with each subject displaying a unique pattern; where beta diversity of the 363 replicate control samples (e.g., labeled in FIG. 5) and the 400 other stool samples was calculated based on genus-level clinical taxa using Bray-Curtis dissimilarity and ordinated using non-metric multidimensional scaling (NMDS) (and/or any suitable analytical techniques can be used for microbiome characterization; etc.); where the 4 insets (e.g., right portion of FIG. 5) show the ordination of the control samples, each showing the same data, colored according to preparation batch, sequencer machine, extraction robot, and operator, respectively.

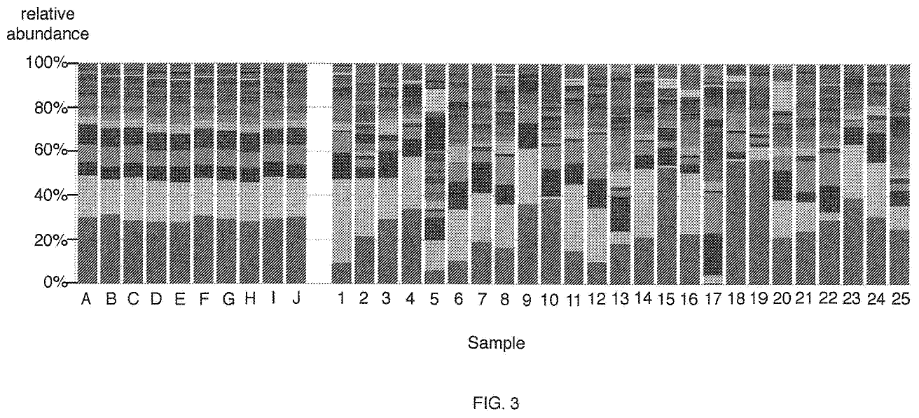

In a specific example, as shown in FIG. 3, control samples (e.g., 10 control samples indicated by samples A through J in FIG. 3; etc.) from a same biological control specimen (e.g., homogenized human stool sample) can be analyzed in different independent microbiome analysis assays (e.g., 10 independent microbiome analysis assays; and/or suitable microorganism-related characterization processes; etc.); where such control sample analyses can be compared to an analysis, using the same sample processing operations (e.g., using same or similar experimental conditions; etc.), of a set of samples from a set of specimens (e.g., 25 different human stool samples, indicated by samples 1-25 in FIG. 3, from 25 different subjects; etc.), where the control sample characterization (e.g., of the 10 control samples, as shown in FIG. 3; etc.) can indicate similar microorganism-related parameters (e.g., similar relative abundance profiles; similar microbiome composition diversity; etc.), and where the multi-specimen analysis (e.g., from different subjects) can indicate different microorganism-related parameters (e.g., different relative abundance profiles; different microbiome composition diversity; etc.).

In specific examples, similarity of microorganism-related parameters across control samples from a single homogenized specimen can motivate the use of such techniques in relation to determining variability parameters for microorganism-related characterization processes (e.g., microbiome assays; etc.).

However, validation, reproducibility analysis, and/or any suitable associated analysis can be performed in any suitable manner.

Determining reference microorganism-related parameters can include determining a set of validator taxa (e.g., a set of taxa to be used, such as in relation to relative abundance values, for comparisons between reference microorganism-related parameters and microorganism-related parameters determined for a control sample characterization associated with a microorganism-related characterization for target samples; etc.). In a specific example, after performing a plurality of microbiome assays with validation control samples, the relative abundance of the 20 most abundant bacterial taxa within each of those validation control samples (e.g., each of 100 replicates, etc.) can be calculated and the average and variations around the average of these 20 taxa can be determined (e.g., for reference cutoff ranges for the validator taxa; etc.). However, any suitable number of validator taxa (e.g., top 10, 15, 20, 25, 30 abundant taxa, etc.) can be used, and can based on any suitable criteria (e.g., relative abundance, absolute abundance, suitable microbiome composition features, suitable microbiome functional features, etc.).

Additionally or alternatively, validator taxa (and/or taxa analyzed in relation to any suitable control sample characterization; and/or taxa used in any suitable portions of embodiments of the method 100 and/or system; etc.) can include taxa characterizable based on 16S gene analysis (e.g., comparison of sequencing read outputs to reference 16S gene sequences corresponding to different microorganism taxa; etc.), taxa associated with any suitable microorganism-related conditions, taxa described in U.S. application Ser. No. 15/707,907 filed 18 Sep. 2017, which is herein incorporated in its entirety by this reference, and/or any suitable microorganism taxa.

In specific examples, microorganism taxa can include any one or more of: Clostridium (genus), Clostridium difficile (species), Alistipes (genus), Alloprevotella (genus), Anaerofilum (genus), Bacteroides (genus), Barnesiella (genus), Bifidobacterium (genus), Blautia (genus), Butyricimonas (genus), Campylobacter (genus), Catenibacterium (genus), Christensenella (genus), Collinsella (genus), Coprococcus (genus), Dialister (genus), Eggerthella (genus), Escherichia-Shigella (genus), Faecalibacterium (genus), Flavonifractor (genus), Fusobacterium (genus), Gelria (genus), Haemophilus (genus), Holdemania (genus), Lactobacillus (genus), Odoribacter (genus), Oscillibacter (genus), Oscillospira (genus), Parabacteroides (genus), Paraprevotella (genus), Peptoclostridium (genus), Phascolarctobacterium (genus), Prevotella (genus), Pseudoflavonifractor (genus), Roseburia (genus), Ruminococcus (genus), Salmonella (genus), Streptococcus (genus), Turicibacter (genus), Tyzzerella (genus), Veillonella (genus), Acetobacter nitrogenifigens (species), Acinetobacter baumannii (species), Akkermansia muciniphila (species), Anaerotruncus colihominis (species), Azospirillun brasilense (species), Bacillus cereus (species), Bacillus coagulans (species), Bacillus licheniformis (species), Bacteroides fragilis (species), Bacteroides vulgatus (species), Bifidobacterium longum (species), Bifidobacterium animalis (species), Bifidobacterium bifidum (species), Brevibacillus laterosporus (species), Butyrivibrio crossotus (species), Campylobacter jejuni (species), Campylobacter coli (species), Campylobacter lari (species), Christensenella minuta (species), Clavibacter michiganensis (species), Clostridium butyricum (species), Collinsella aerofaciens (species), Coprococcus eutactus (species), Desulfovibrio piger (species), Dialister invisus (species), Enterococcus italicus (species), Escherichia coli (species), Escherichia coli O157 (species), Faecalibacterium prausnitzii (species), Fibrobacter succinogenes (species), Kocuria rhizophila (species), Lactobacillus brevis (species), Lactobacillus coryniformis (species), Lactobacillus delbrueckii (species), Lactobacillus fermentum (species), Lactobacillus helveticus (species), Lactobacillus kefiranofaciens (species), Lactobacillus kunkeei (species), Lactobacillus rhamnosus (species), Lactobacillus salivarius (species), Lactococcus fujiensis (species), Lactococcus garvieae (species), Lactococcus lactic (species), Leptotrichia hofstadii (species), Leuconostoc fallax (species), Leuconostoc kimchii (species), Methanobrevibacter smithii (species), Oenococcus oeni (species), Oxalobacter formigenes (species), Paenibacillus apiarius (species), Pediococcus pentosaceus (species), Peptoclostridium difficile (species), Propionibacterium freudenreichii (species), Pseudoclavibacter helvolus (species), Renibacterium salmoninarum (species), Ruminococcus albus (species), Ruminococcus flavefaciens (species), Ruminococcus bromii (species), Ruminococcus gnavus (species), Salmonella bongori (species), Salmonella enterica (species), Shigella boydii (species), Shigella sonnei (species), Shigella flexneri (species), Shigella dysenteriae (species), Staphylococcus sciuri (species), Streptococcus sanguinis (species), Streptococcus thermophilus (species), Vibrio cholerae (species), Weissella koreensis (species), Yersinia enterocolitica (species)

Determining one or more reference microorganism-related parameters is preferably based on an analyzing one or more control samples. Analyzing one or more control samples (and/or any suitable portions of embodiments of the method 100 and/or system) can include any one or more of: sequencing operations, alignment operations (e.g., sequencing read alignment; etc.), lysing operations, cutting operations, tagging operations (e.g., with barcodes; etc.), ligation operations, fragmentation operations, amplification operations (e.g., helicase-dependent amplification (HDA), loop mediated isothermal amplification (LAMP), self-sustained sequence replication (3SR), nucleic acid sequence based amplification (NASBA), strand displacement amplification (SDA), rolling circle amplification (RCA), ligase chain reaction (LCR), etc.), purification operations, cleaning operations, homogenization processes, heterogenization processes, aliquoting processes, replicate sample preparation processes, suitable operations for sequencing library preparation, suitable operations for facilitating sequencing and/or downstream analysis, suitable sample processing operations, and/or any suitable sample- and/or sequence-related operations. In variations, determining one or more reference microorganism-related parameters can include: generating a sequencing library (e.g., through multi-step PCR amplification processes; through metagenome sequencing library processes; through amplicon sequencing library processes; through fragmentation processes; etc.), such as based on one or more control samples; sequencing the sequencing library (e.g., with a next generation sequencing system and/or any suitable sequencing technology; etc.); and determining one or more reference microorganism-related parameters based on outputs of the sequencing (e.g., based on sequence read alignments between the sequencing reads and reference 16S sequences corresponding to different microorganism taxa, such as to determine absolute and/or relative abundances of different microorganism taxa in the control sample; etc.). In variations, determining one or more reference microorganism-related parameters can include performing any suitable processes described in and/or analogous to U.S. application Ser. No. 15/707,907 filed 18 Sep. 2017, which is herein incorporated in its entirety by this reference.

Determining one or more reference microorganism-related parameters (and/or suitable portions of embodiments of the method 100, etc.) preferably includes processing one or more control samples in a manner associated with (e.g., analogous to; similar to; the same as; etc.) processing for a microorganism-related characterization (e.g., for characterizing a target, sample, such as for determining a characterization for a microorganism-related condition; etc.). In specific examples, processing the one or more control samples can be performed in a manner that will be performed for subsequent (and/or performed at any suitable time) microorganism-related characterization assays (e.g., assays for characterization of target samples from new users to be characterized; etc.). In a specific example, processing control samples can include facilitating control sample processing of the control sample in a manner associated with target sample processing (e.g., earlier processing, concurrent processing, future processing, etc.) of target samples corresponding to the microorganism-related characterization process. Additionally or alternatively, processing one or more control samples (e.g., to determine reference microorganism-related parameters; for control sample characterization included in a microorganism-related characterization process; etc.) can be performed in any suitable manner (e.g., same as, similar to, or different from processing of target samples in a microorganism-related characterization process, such as a microorganism-related characterization process to be evaluated in relation to variability; etc.).

In variations, reference microorganism-related parameters can include and/or be associated with microorganism functionality (e.g., values for gene expression associated with microorganism functionality; microbiome functional features; etc.). In variations, determining reference microorganism-related parameters can include determining validator functions (e.g., a set of microorganism functions, etc.), such as where values for different validator functions can be used as reference microorganism-related parameters (e.g., for comparison to analogous values for microorganism function determined in control sample characterization, such as control sample characterization determined in association with microorganism-related characterization processes; etc.).

Additionally or alternatively, determining microorganism-related parameters, determining control sample characterizations, determining microorganism-related characterizations, and/or suitable portions of embodiments of the method 100 and/or system, can include, apply, employ, perform, use, be based on, and/or otherwise be associated with one or more analytical techniques including any one or more of: extracting features (e.g., microbiome composition features; microbiome functional features; etc.), performing pattern recognition on data, fusing data from multiple sources, combination of values (e.g., averaging values, etc.), determining variation (e.g., standard deviation calculations; variability calculations, such as based on averages; etc.) compression, conversion, performing statistical estimation on data, normalization, updating, ranking, weighting, validating, filtering (e.g., for baseline correction, data cropping, etc.), noise reduction, smoothing, filling, aligning, model fitting, binning, windowing, clipping, transformations, mathematical operations (e.g., derivatives, moving averages, summing, subtracting, multiplying, dividing, etc.), data association, interpolating, extrapolating, clustering, visualizing, and/or any other suitable processing operations.

However, determining reference microorganism-related parameters S120 can be performed in any suitable manner.

### 2.3 Determining a Variability Parameter.

Embodiments of the method 100 can include determining one or more variability parameters S130, which can function to describe, indicate, evaluate, analyze, and/or otherwise characterize one or more microorganism-related characterization processes, such as via a control sample characterization for a control sample processed (e.g., along with one or more target samples, etc.) in the microorganism-related characterization process.

Variability parameters preferably characterize (e.g., describe, indicate aspects regarding; etc.), one or more microorganism-related characterization processes (e.g., microbiome assays; sample processing operations; sequencing operations; bioinformatics operations; microorganism-related processes; etc.), but can additionally or alternatively characterize any suitable aspects. Variability parameters can include one or more of: classifications (e.g., labeling of microbiome assay runs and/or suitable microorganism-related characterization processes as a “pass” or “fail”, such as based on comparisons between one or more reference microorganism-related parameters and control sample characterizations; etc.); individual values (e.g., individual variability parameters for individual control samples ran with one or more target samples in a microorganism-related characterization process such as a microbiome assay; etc.); overall values (e.g., averages, median, aggregate, and/or combined individual variability parameters, such as an overall determination of a “pass” or “fail” for a microorganism assay run and/or microorganism-related characterization process, such as based on individual “pass” or “fail” values; etc.); ranges (e.g., confidence metrics associated with one or more variability parameters; quality ranges; etc.), standard deviations (e.g., in relation to averages; etc.); absolute values; changes in values (e.g., changes in variability parameters over times; changes in variability parameters across control samples; changes in variability parameters across experimental conditions; etc.); verbal indications (e.g., “pass”, “fail”, “high quality”, “medium quality”, “low quality”, etc.); numerical indications (e.g., quality scores; number of taxa passing one or more thresholds; etc.); and/or any suitable forms of variability parameters.

In examples, determining one or more variability parameters can include determining variability parameters describing quality (e.g., in relation to variability and/or reproducibility; etc.) of a microbiome assay run (e.g., including assaying of one or more target samples associated with one or more users, along with assaying of one or more control samples; etc.), such as in order to determine the usability of the microbiome assay run. In a specific example, determining one or more variability parameters can include classifying the microbiome assay run as a “pass” or “fail” (e.g., based on degree of deviation of relative abundance values of the control sample of the microbiome assay run for a set of taxa in relation to reference cutoff ranges for the set of taxa and/or other suitable reference microorganism-related parameters; etc.). However, variability parameters can be configured in any suitable manner.

In a specific example, the microorganism-related characterization process can include a microbiome assay associated with diagnostics for a microorganism-related condition, and where determining the variability parameter can be for the microbiome assay. In a specific example, the microbiome assay can correspond to a microbiome assay type, where the first processing operations (e.g., for processing a set of validation control samples, for determining reference microorganism-related parameters; such as in relation to S120; etc.) can include a set of validation microbiome assays corresponding to the microbiome assay type (e.g., a same microbiome assay type as a microbiome assay used in assaying one or more target samples; etc.) and performed with the first subset of control samples, and where the microbiome assay is performed with the second subset of control samples and the target sample from the user (e.g., performed with the same experimental conditions for assaying the second subset of control samples along with one or more target samples from the user; etc.). In a specific example, the variability parameter can describe quality of one or more microbiome assays (e.g., the microbiome assay performed with the second subset of control samples and the target sample from the user; etc.), and where determining the variability parameter can include determining the variability parameter for the microbiome assay based on a deviation (e.g., degree of deviation) between the control sample characterization and the set of reference microorganism abundance parameters. In a specific example, determining the variability parameter can include determining a passing metric for the microbiome assay if the deviation satisfies a threshold condition; and determining a failing metric for the microbiome assay if the deviation fails the threshold condition.

Control sample characterizations preferably characterize (e.g., describe, indicate aspects regarding; etc.) one or more control samples in relation to microorganisms (e.g., microorganism composition; microorganism function; etc.) from the control sample, but can additionally or alternatively characterize and/or be associated with any suitable aspects. Control sample characterizations preferably include one or more microorganism-related parameters (e.g., describing and/or indicating one or more suitable microorganism-related aspects of the one or more control samples; etc.), but can additionally or alternatively include any suitable data (e.g., control sample identifiers; control sample metadata; etc.) and/or components. Microorganism-related parameters of control sample characterizations are preferably of the same type and/or form of reference microorganism-related parameters (e.g., for comparison between the microorganism-related parameters and reference microorganism-related parameters; etc.). In specific examples, reference microorganism-related parameters can include reference cutoff ranges of relative abundance for a set of taxa (e.g., validator taxa; etc.), and microorganism-related parameters can include relative abundance values for the set of taxa (e.g., for determination as to whether, for each set of taxa, the relative abundance values fall in the reference cutoff ranges; etc.). Microorganism-related parameters can include any suitable type and/or form of reference microorganism-related parameters (e.g., described herein; etc.). In specific examples, microorganism-related parameters (e.g., of control sample characterizations, etc.) can include microorganism abundance parameters, microbiome composition parameters (e.g., microbiome composition data; microbiome composition features; microbiome composition diversity; etc.); microorganism function parameters (e.g., associated with microorganism function; microbiome functional data; microbiome functional features; microbiome functional diversity; etc.); and/or any suitable microorganism-related parameters. Microorganism-related parameters can be of any suitable forms, including any one or more of: individual values (e.g., for a control sample ran with one or more target samples in a microorganism-related characterization process such as a microbiome assay; individual relative and/or absolute abundance values for different taxa for a control sample; etc.); overall values (e.g., averages for relative and/or absolute abundance values for different taxa, such as determined from averaging individual values for different control samples ran with one or more target samples in a microorganism-related characterization process; etc.); ranges (e.g., ranges around averages and/or medians for relative and/or absolute abundance of a given microorganism taxon; etc.), standard deviations (e.g., in relation to averages, for use in determining ranges; etc.); absolute values (e.g., absolute counts for a microorganism taxon, etc.); changes in values (e.g., changes in relative abundance over time from a series of control samples over time, such as a series of control samples used in a series of microorganism-related characterization processes such as with a series of target samples from monitoring a user over time in relation to one or more microorganism-related conditions; changes in any suitable parameters over time; changes in suitable parameters across experimental conditions, such as in relation to different operators, different experimental runs, and/or other suitable conditions; etc.); and/or any suitable forms of reference microorganism-related parameters. However, control sample characterizations can be configured in any suitable manner.

Determining one or more variability parameters (and/or suitable portions of embodiments of the method 100, etc.) preferably includes processing one or more control samples in a manner associated with (e.g., analogous to; similar to; the same as; etc.) processing one or more target samples for a microorganism-related characterization (e.g., such as for determining a characterization for a microorganism-related condition based on analysis of the one or more target samples; etc.). In specific examples, processing the one or more control samples can be performed in the same microbiome assay (and/or including sample preparation for the microbiome assay; etc.) as used for assaying of the one or more target samples, such as where the one or more control samples and the one or more target samples undergo similar or same experimental conditions (e.g., for enabling monitoring and/or capturing of variations within the experimental processes; etc.).

Determining one or more variability parameters preferably includes comparing one or more control sample characterizations (e.g., microorganism-related parameters for control samples; etc.) with one or more reference microorganism-related parameters. In a specific example, determining one or more variability parameters can include determining if a predefined portion of the selected microbial taxa (e.g., validator taxa; etc.), for one or more control samples associated with the microorganism-related characterization process (e.g., processed with one or more target samples; etc.), fall within a prespecified range (e.g., reference microorganism-related parameter; etc.) around the average (mean) relative abundance of the taxa using the same calculations as described above. In a specific example, for each validation taxon, the relative abundance for one or more control samples (e.g., ran with target samples in a microbiome assay to be used for microorganism-related characterization for a user; etc.) can be compared to a reference relative abundance parameter (e.g., average, determined from a set of validation control samples prepared from the same individual specimen as one or more control samples ran with one or more target samples; etc.). In specific examples, deviations from reference microorganism-related parameters (e.g., mean) can indicate the quality of a microbiome assays; such as where a large deviation can reflect a poor-quality run, while a value close to the average can reflect high-quality run. In specific examples, a control sample (e.g., ran with one or more target samples; etc.) can pass the quality control if it has a score of equal to, or above, a predefined cutoff (and/or suitable reference microorganism-related parameters; etc.). In a specific example, to check the quality of a new assays (and/or suitable microorganism-related characterization process; etc.), the relative abundance of each validation taxon (e.g., for a control sample ran with one or more target samples; etc.) can be compared to the calculated average of that validation taxon; where if the relative abundance of a particular validation taxon falls within a predefined range around the mean (e.g., within 2 times the standard variation, or as calculated by beta distribution, and/or any other range), the score for that taxon will be 1; where the scores of each taxon can then be summed; where the maximum score for a control sample thus is equal to the number of validation taxa chosen (e.g., 20, etc.), while the minimum score is 0; and where a control sample then will pass the quality control if the score (e.g., number of taxa for which relative abundance values fall into the reference ranges' etc.) equal to, or above, a predefined cut off (e.g., cutoff score of 17, indicating relative abundance values fell into reference ranges for 17 or more taxa, such as out of 20 taxa; etc.).

In variations, comparing a control sample characterization with reference microorganism-related parameters can include comparing microorganism function parameters (e.g., for a control sample ran with target samples in a microbiome assay; etc.) with reference microorganism function parameters (e.g., reference microorganism function ranges; etc.). However, comparing control sample characterizations and reference microorganism-related parameters can be performed in any suitable manner. In a specific example, determining a set of reference microorganism-related parameters can be based on the processing operations associated with the microorganism-related characterization process (e.g., processing operations using same or similar experimental conditions as the microorganism-related characterization process; etc.). In a specific example, determining the set of reference microorganism-related parameters can include determining a set of reference microorganism-related ranges based on the processing operations with a subset of the set of control samples (e.g., a set of control samples derived from the same individual specimen; etc.), and where determining the variability parameter can include determining the variability parameter based on the comparison between the control sample characterization and the set of reference microorganism-related parameters. In a specific example, the set of reference microorganism-related parameters can include a set of reference microorganism function parameters, where the control sample characterization can include a set of microorganism function parameters for at least one control sample (e.g., one or more control samples processed with one or more target samples in one or more microorganism-related characterization processes; etc.), and where determining the variability parameter can include determining the variability parameter based on the comparison between the set of microorganism function parameters and the set of reference microorganism function parameters. In a specific example, the set of reference microorganism-related parameters can further include (e.g., in addition to reference microorganism function parameters; etc.) a set of reference microorganism abundance parameters, where the control sample characterization can further include (e.g., in addition to reference microorganism function parameters; etc.) a set of microorganism abundance parameters for the at least one control sample, and where determining the variability parameter can include determining the variability parameter based on the set of microorganism abundance parameters, the set of reference microorganism abundance parameters, the set of microorganism function parameters, and the set of reference microorganism function parameters (e.g., determining whether abundance parameters fall into reference abundance parameter rangers; determining whether function parameters fall into reference function parameter ranges; etc.).

Determining variability parameters, any suitable portions of embodiments of the method 100, and/or suitable portions of embodiments of the system, can include, apply, employ, perform, use, be based on, and/or otherwise be associated with artificial intelligence approaches (e.g., machine learning approaches, etc.) including any one or more of: supervised learning (e.g., using logistic regression, using back propagation neural networks, using random forests, decision trees, etc.), unsupervised learning (e.g., using an Apriori algorithm, using K-means clustering), semi-supervised learning, a deep learning algorithm (e.g., neural networks, a restricted Boltzmann machine, a deep belief network method, a convolutional neural network method, a recurrent neural network method, stacked auto-encoder method, etc.), reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminant analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a dimensionality reduction method (e.g., principal component analysis, partial least squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and/or any suitable artificial intelligence approach. In variations, determining variability parameters can be based on a variability parameter model (e.g., machine learning model), such as a variability parameter model (e.g., trained on reference microorganism-related parameters; etc.) used to classify one or more microorganism-related characterization processes (e.g., as “pass” or “fail”) based on inputs from one or more control sample characterizations (e.g., relative abundance values for a control sample ran with target samples in the one or more microorganism-related characterization processes; etc.). However, artificial intelligence approaches can be configured and/or applied in any suitable manner.

However, determining variability parameters S130 can be performed in any suitable manner.

### 4. Other

Embodiments of the method 100 can, however, include any other suitable blocks or steps configured to facilitate reception of biological samples from subjects, processing of biological samples from subjects, analyzing data derived from biological samples, and generating models that can be used to provide customized diagnostics and/or probiotic-based therapeutics according to specific microbiome compositions and/or functional features of subjects.

Embodiments of the method 100 and/or system can include every combination and permutation of the various system components and the various method processes, including any variants (e.g., embodiments, variations, examples, specific examples, figures, etc.), where portions of embodiments of the method 100 and/or processes described herein can be performed asynchronously (e.g., sequentially), concurrently (e.g., in parallel), or in any other suitable order by and/or using one or more instances, elements, components of, and/or other aspects of the system and/or other entities described herein.

Any of the variants described herein (e.g., embodiments, variations, examples, specific examples, figures, etc.) and/or any portion of the variants described herein can be additionally or alternatively combined, aggregated, excluded, used, performed serially, performed in parallel, and/or otherwise applied.

Portions of embodiments of the method 100 and/or system can be embodied and/or implemented at least in part as a machine configured to receive a computer-readable medium storing computer-readable instructions. The instructions can be executed by computer-executable components that can be integrated with the system. The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the method 100, system, and/or variants without departing from the scope defined in the claims.

We claim:

1. A method for improving a microorganism-related characterization process, the method comprising:
preparing a set of control samples from an individual specimen;
determining a set of reference microorganism abundance parameters based on first processing operations with a first subset of control samples from the set of control samples, wherein the first processing operations are associated with the microorganism-related characterization process;
determining a control sample characterization for a second subset of control samples from the set of control samples, based on the microorganism-related characterization process with the second subset of control samples and a target sample from a user; and
determining a variability parameter for the microorganism-related characterization process based on a comparison between the control sample characterization and the set of reference microorganism abundance parameters.

2. The method of claim 1, wherein determining the set of reference microorganism abundance parameters comprises:
determining a set of individual reference microorganism abundance parameters based on the first processing operations with the first subset of control samples; and
determining a set of average reference microorganism abundance parameters based on the set of individual reference microorganism abundance parameters,
wherein determining the variability parameter comprises determining the variability parameter based on the control sample characterization and the set of average reference microorganism abundance parameters.

3. The method of claim 2, wherein determining the set of reference microorganism abundance parameters comprises determining a set of reference microorganism abundance ranges based on the set of average reference microorganism abundance parameters, and wherein determining the variability parameter comprises determining the variability parameter based on the comparison between the control sample characterization and the set of reference microorganism abundance ranges.

4. The method of claim 3, wherein determining the set of reference microorganism abundance ranges comprises determining the set of reference microorganism abundance ranges for a set of validator microorganism taxa, wherein the control sample characterization comprises a set of microorganism abundance parameters for the set of validator microorganism taxa, and wherein determining the variability parameter comprises, for each taxon of the set of validator microorganism taxa, determining whether a corresponding microorganism abundance parameter of the set of microorganism abundance parameters is in a corresponding reference microorganism abundance range of the set of reference microorganism abundance ranges.

5. The method of claim 4, wherein determining the variability parameter comprises:
determining a taxa-related score based on a number of taxa with the corresponding microorganism abundance parameters in the corresponding reference microorganism abundance ranges; and
determining the variability parameter based on a comparison between the taxa-related score and a taxa-related score threshold.

6. The method of claim 5, further comprising determining the taxa-related score threshold based on a set of criteria associated with maximization of control sample passing rate and minimization of non-control sample passing rate.

7. The method of claim 3, wherein the set of reference microorganism abundance ranges comprises a set of reference microorganism relative abundance ranges, wherein the control sample characterization comprises a set of microorganism relative abundance parameters, and wherein determining the variability parameter comprises determining the variability parameter based on the comparison between the set of microorganism relative abundance parameters and the set of reference microorganism relative abundance ranges.

8. The method of claim 1, further comprising:
determining a therapy for the user for a microorganism-related condition based on the variability parameter for the microorganism-related characterization process; and
facilitating provision of the therapy to the user.

9. The method of claim 8, wherein facilitating provision of the therapy comprises providing the therapy to the user.

10. The method of claim 1, wherein the microorganism-related characterization process comprises a microbiome assay associated with diagnostics for a microorganism-related condition, and wherein determining the variability parameter comprises determining the variability parameter for the microbiome assay.

11. The method of claim 10, wherein the microbiome assay corresponds to a microbiome assay type, wherein the first processing operations comprises a set of validation microbiome assays corresponding to the microbiome assay type and performed with the first subset of control samples, and wherein the microbiome assay is performed with the second subset of control samples and the target sample from the user.

12. The method of claim 10, wherein the variability parameter describes quality of the microbiome assay with the second subset of control samples and the target sample from the user, and wherein determining the variability parameter comprises determining the variability parameter for the microbiome assay based on a deviation between the control sample characterization and the set of reference microorganism abundance parameters.

13. The method of claim 12, wherein determining, the variability parameter comprises: determining a passing metric for the microbiome assay if the deviation satisfies a threshold condition; and determining a failing metric for the microbiome assay if the deviation fails the threshold condition.

14. The method of claim 1, wherein preparing the set of control samples from the individual specimen comprises:
homogenizing the individual specimen in relation to microorganism content from the individual specimen; and
aliquoting the homogenized individual specimen.

15. The method of claim 14, wherein the individual specimen comprises a stool sample, wherein homogenizing the individual specimen comprises mixing the stool sample with a saline solution.

16. A method for improving a microorganism-related characterization process, the method comprising:
preparing a set of control samples;
determining a control sample characterization for at least one control sample of the set of control samples, based on the microorganism-related characterization process with the at least one control sample and a target sample; and
determining a variability parameter for the microorganism-related characterization process based on a comparison between the control sample characterization and a reference microorganism-related parameter determined from processing operations associated with the microorganism-related characterization process.

17. The method of claim 16, further comprising determining a set of reference microorganism-related parameters comprising the reference microorganism-related parameter, based on the processing operations associated with the microorganism-related characterization process.

18. The method of claim 17, wherein determining the set of reference microorganism-related parameters comprises determining a set of reference microorganism-related ranges based on the processing operations with a subset of the set of control samples, and wherein determining the variability parameter comprises determining the variability parameter based on the comparison between the control sample characterization and the set of reference microorganism-related parameters.

19. The method of claim 17, wherein the set of reference microorganism-related parameters comprises a set of reference microorganism function parameters, wherein the control sample characterization comprises a set of microorganism function parameters for the at least one control sample, and wherein determining the variability parameter comprises determining the variability parameter based on the comparison between the set of microorganism function parameters and the set of reference microorganism function parameters.

20. The method of claim 19, wherein the set of reference microorganism-related parameters further comprises a set of reference microorganism abundance parameters, wherein the control sample characterization further comprises a set of microorganism abundance parameters for the at least one control sample, and wherein determining the variability parameter comprises determining the variability parameter based on the set of microorganism abundance parameters, the set of reference microorganism abundance parameters, the set of microorganism function parameters, and the set of reference microorganism function parameters.

21. The method of claim 16, wherein the reference microorganism-related parameter comprises a reference microorganism relative abundance parameter for a microorganism taxon, wherein the control sample characterization comprises a microorganism relative abundance parameter for the microorganism taxon, and wherein determining the variability parameter comprises determining the variability parameter based on the comparison between the microorganism relative abundance parameter and the reference microorganism relative abundance parameter.

22. The method of claim 16, wherein preparing a set of control samples comprises preparing a set of control samples from an individual specimen, and wherein preparing the set of control samples from the individual specimen comprises homogenizing the individual specimen.

23. The method of claim 16, further comprising:
determining a therapy for a user for a microorganism-related condition based on the variability parameter for the microorganism-related characterization process; and facilitating provision of the therapy to the user.
